(19) United States
(12) Patent Application Publication
(10) Pub. No.: US 2007/0183924 A1
(43) Pub. Date: Aug. 9, 2007

(54) Portable device for vapour emanation by heating a volatile compound in a cartridge

(75) Inventor: John Douglas Peter Morgan, Carlingford (AU)

Correspondence Address: CONLEY ROSE, P.C.;David A. Rose, P. O. BOX 3267, HOUSTON, TX, 77253-3267, US

(73) Assignee: Reckitt Benckiser Pty Limited, West Ryde (AU)

(21) Appl. No.: 10/565,055

(22) PCT Filed: Jul. 16, 2004
(86) PCT No.: PCT/GB04/03083

§ 371 Date: Jul. 11, 2006

(30) Foreign Application Priority Data
Jul. 18, 2003 (GB) 0316803.6

(51) Int. Cl. A61L 9/00; A62B 7/08; A01M 21/04; B05B 5/053

(52) U.S. Cl. 422/5; 422/125; 47/1.5; 361/226

(57) ABSTRACT

A device for enabling vapourization of a chemical formulation into an atmosphere comprising means (4) for receiving a cartridge including the chemical formulation, aperture means (3) to enable vapourized chemical formulation to be guided therethrough into the atmosphere, wherein the chemical formulation contacts a heater means in order to vapourize the chemical formulation.

### FIELD OF THE INVENTION

This invention relates to a device, particularly a portable device, that provides for the emanation of a chemical formulation, such as an insecticide or a fragrance, into a surrounding atmosphere.

### BACKGROUND TO THE INVENTION

A number of patent documents disclose portable devices that enable the application of a herbicide or an insecticide, particularly for the purpose of killing weeds and the like. Such an example is disclosed in U.S. Pat. No. 4,309,842 in which a portable hand-held herbicide and insecticide applicator comprises a tube 12 which acts as a handle and connected at one end of the handle is a pair of hollow prong sections. The prong sections together with the handle 12 form a liquid reservoir conduit for the pesticide or herbicide. Extending between the ends of the prong sections is an absorptive rope mop which is saturated with the liquid pesticide or herbicide. The user then drags the device over weeds and the like such that the saturated rope mop comes into contact with the weeds and thereby eventually kills the weeds.

Other devices include sprayer systems that have a reservoir and use an electric motor, such as is disclosed in U.S. Pat. No 6,109,548. This sprayer system reduces the need to manually create pressure within a portable spray unit. A valve stem within the reservoir may be utilised by a user to connect a conventional air tank thereto for inserting high amounts of air pressure directly into the reservoir without requiring the use of a manual or electric pump. Where a power switch is closed the electric motor operates the air compressor whenever the air pressure within the reservoir drops below a predetermined level. A manual pump may be utilised when the electric pump fails or a battery connected to the electric motor becomes discharged.

Other systems involve the atomisation of liquids, such as is disclosed in U.S. Pat. No 4,356,528. This patent discloses an invention that utilises electrostatic spraying of liquid compositions, particularly over leaves of plants, by supplying a liquid to a spray orifice, which is preferably of capillary dimensions. The spray orifice has a charged surface which is electrically conducting or semi-conducting and is adjacent a field intensifying electrode. The arrangement being such that the liquid is drawn out primarily by electrostatic forces, atomised into electrically charged particles and projected past the electrode. Thus the particles wrap around the leaves of the plants and coat both upper and lower surfaces of the leaves.

None of the above prior art systems provide a portable, substantially low cost personal device that enables the discharge into surrounding atmosphere of a chemical formulation, particularly for the purpose of a personal insect repellent or in situations where a portable small device may be used in a room which periodically enables the emanation or vaporisation of the chemical formulation. There is a need for such a device that is substantially low cost to run, may be run on batteries and last a substantial length of time without having to replace batteries or provide a new or refilled reservoir of the chemical formulation.

The present invention has substantial advantages over the above mentioned applicators and sprayers and conventional aerosol sprayers as there is no need for the user to physically operate the device as it may be left in an area to automatically discharge a chemical vapour into surrounding atmosphere.

### SUMMARY OF THE INVENTION

According to a first aspect of the invention there is provided a portable device for enabling vapourization of a chemical formulation into an atmosphere comprising:

means for receiving a cartridge, the cartridge storing the chemical formulation;

heater means for contacting the chemical formulation and vapourizing the chemical formulation when the cartridge is inserted into the receiving means and the heater means is energised;

aperture means to enable vapourized chemical formulation to be guided therethrough into the atmosphere.

Preferably the receiving means is a recess having an opening. The recess may have guide means for guiding the cartridge when the cartridge is inserted into the recess.

The recess or guide means may have projection means, preferably in the form of one or more lugs, that provide an interference fit with corresponding indentation means, preferably in the form of one or more notches, on the cartridge in order to locate the cartridge in a fixed relationship with respect to the heater means.

Alternatively the recess or guide means may have indentation means, preferably in the form of one or more notches, that provide an interference fit with corresponding projection means, preferably in the form of one or more lugs, on the cartridge in order to locate the cartridge in a fixed relationship with respect to the heater means.

The device may include electrical circuit means in order to provide one or more pulses sequentially to the heater means so as to enable the heater means to vapourize the chemical formulation. The device may have compartment means to accommodate a power supply, preferably in the form of batteries in order to provide power to the electrical circuit means.

The device may further comprise a partition that divides the interior of the device into a first compartment and a second compartment. The partition may comprise an electrical circuit board housing an electrical circuit means. The electrical circuit board may be double-sided providing electrical connections to electrical components of the electrical circuit means. The electrical components of the electrical circuit means may be located on one or both sides of the electrical circuit board.

The heater means may be mounted on a side of the electrical circuit board within the first compartment, the first compartment preferably housing the receiving means and having the aperture means so as to enable the heater means to contact the chemical formulation on the cartridge when the cartridge is inserted into the receiving means.

The device may be constructed as two detachable members, a first or top member housing the receiving means and aperture means, and a second or base member preferably housing the electrical circuit means and power supply. Alternatively the device may be constructed as a single article. The heater means is preferably in the form of a microheater element and more particularly in the form of an impedance means such as a resistor. Alternatively, the first and second members may be divided by a partition forming an electrical circuit board for the electrical circuit means. Preferably the electrical circuit board for the electrical circuit means is double-sided and has mounted on one side the heater means such that the heater means is in communication with components of the electrical circuit means on the opposite side, but protrudes into the top member to enable contact with the chemical formulation on the cartridge when the cartridge is inserted into the receiving means, preferably through a wick means. Alternatively, some or all of the components of the electrical circuit means, such as an indicator means and switch means, are located on the same side of the electrical circuit board, preferably on the side protruding into the top or first member.

The device may have a switch means to activate and deactivate the power supply and therefore the emanation of the chemical formulation. The device may further have indicator means to indicate battery charge status and/or device failure.

### BRIEF DESCRIPTION OF THE DRAWINGS

Preferred embodiments of the invention will hereinafter be described, by way of example only, with reference to the accompanying drawings wherein:

FIG. 1 is a perspective view of a device in accordance with the present invention;

FIG. 2 is an underneath view of the device of FIG. 1 with a member cover removed;

FIG. 3 is a plan view of the device of FIG. 2;

FIG. 4 is an end view of the device of FIG. 1; and

FIG. 5 is a cross sectional side view of the device of FIG. 1 showing guide means for a cartridge.

### DETAILED DESCRIPTION OF PREFERRED EMBODIMENTS

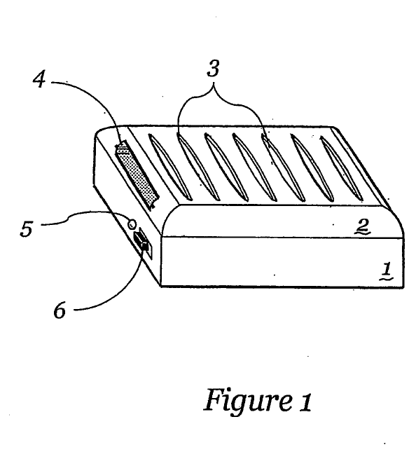

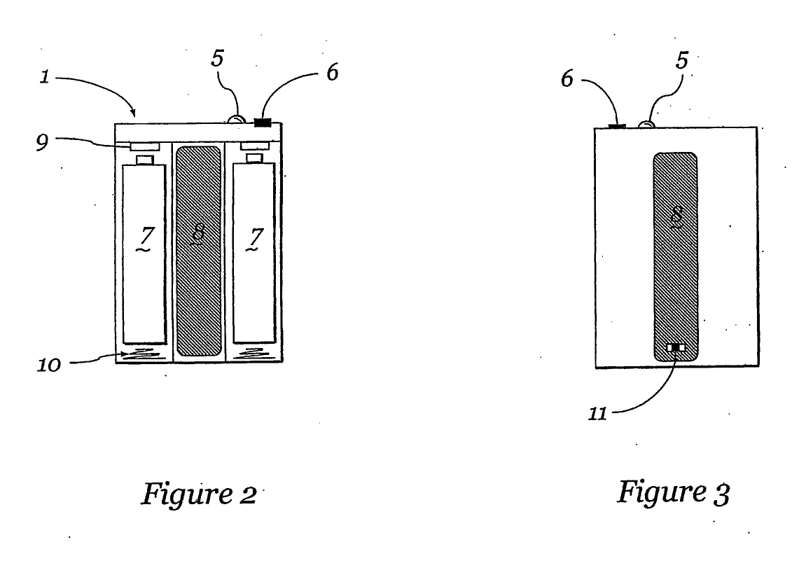

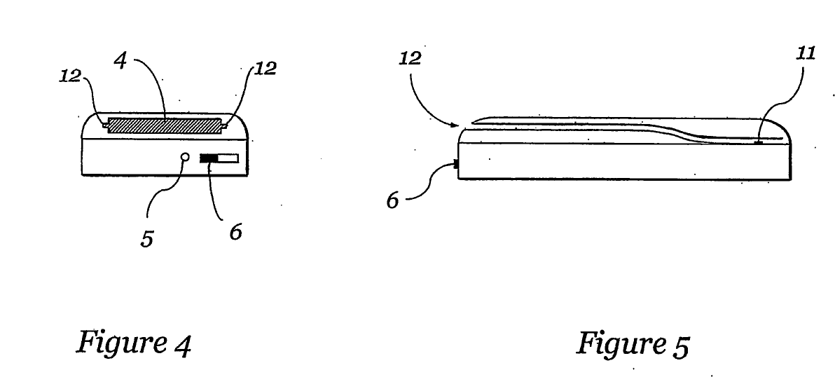

Shown in FIG. 1 is a portable device which may be carried by a user or placed in, for example, a room or another location. The device comprises a first or top member 2 and a second or bottom member 1. The second member 1 houses a pair of batteries 7 and an electrical circuit board 8 which is mounted flush with the top surface of the member 1. The electrical circuit board 8 (PCB) may be double-sided and form a partition or dividing panel between the first member 2 and the second member 1. Electrical components forming an electrical circuit means may be located on a first side, a second side or both sides of the board 8. It is to be noted that the device in an alternative arrangement may be constructed in a single piece such as through injection moulding rather than two separate members 1 and 2. In one embodiment, all electronic components and connections are mounted on the lower side of the circuit board 8, in other words within or facing the interior of the member 1. A heater means 11 is located on the upper side of the electrical circuit board 8 mounted flush with the upper or top surface, such that the heater means 11 protrudes into member 2 to be in contact with chemical formulation. In another embodiment, the heater means 11 , a switch 6 and an indicator 5 are mounted on the same side of the electrical circuit board 8. Where the device is a singular construction or formed of two detachable members 2 and 1, a partition forming either a single or double-sided electrical circuit board 8 may divide the device into two compartments. The first compartment may house a recess or receiving means (see FIGS. 4 and 5) and have apertures 3 while the second compartment houses the power supply 7. The heater means 11 is preferably in the form of a microheater element and more particularly an impedance means such as a resistor. The batteries 7 supply power to the electrical circuit board components which in turn deliver a series of pulses to the heater means 11 in order to heat the heater means 11. The batteries are mounted in such a manner that the positive end of each battery 7 connects to a positive battery terminal 9 and the negative end of each battery 7 connects to a negative battery terminal 10. Typically AA batteries are used but any other form or size of batteries may be used, such as AAA batteries.

The lower member 1 also has a switch means 6 used to turn the device on and off and to allow the user to control or provide additional bursts of vapour when initially turning the device on. Alternatively there may be a further mode, such as a high burst mode, which the user can move the switch to in order to provide additional bursts of vapour. Pulses maybe supplied to the heater means 11 via a suitable power supply.

Also shown adjacent the switch means 6 is an indicator 5 to provide an indication to the user where, for example, the resistor or microheater element 11 burns out or where the batteries are running low. When used to indicate the resistor 11 burning out or having some other fault, the indication might be provided by the light not turning off if a high resistance is sensed. A battery OK indicator may be instituted by using a LED indicator turning on for some seconds, then off, or beeping if the battery is satisfactory. Alternatively the indicator may be used as a LED or a buzzer or an indication scheme analogous to car indicator lights, for example, a correct device operation as indicated by ON for several seconds then OFF when device is first turned on. Failure to light means battery failure and failure to go OFF means the device or resistor has failed in some manner. All of these indicators are necessary as the effect of the product is invisible in terms of the fragrance or insecticide being released and, as it is also inaudible the only indication of a flat battery or a blown microheater element 11 will mean that the consumer suffers bites or is not aware of any fragrance.

The first member 2, which may be detachable from the lower or first member 1, has a series of apertures 3, forming a vent, and an opening 4 for receiving a cartridge type device which provides chemical formulation, such as insecticide or fragrance. Specifically the cartridge has a reservoir which contains the insecticide or fragrance and has a wick which is in constant contact with the insecticide or fragrance such that the chemical formulation is transferred through the wick from one end to the other end of the wick, with the other end of the wick being in contact with or close to a microheater element. Such a cartridge device is described in copending British patent application numbers 0317183.2 and 0410431.1 to the present applicant. When the heater element heats up it thereby vaporises the chemical formulation in the wick in the vicinity of the heater and is dispersed through the series of apertures 3 into the surrounding atmosphere. The opening 4 provides entry to a recess which has guides 12 on either side of the recess (see FIGS. 4 and 5), into which the edges of the cartridge fit thereby guiding the cartridge through the recess towards the inner end of the recess. Adjacent the inner end of the recess is located the microheater element 11. The recess additionally has a pair of projections or lugs, one on each side, that provide an interference fit with corresponding notches or indentations in the cartridge, one on each side. Once the notches fit with the lugs it provides an indication to the user, through tactile feedback, that the cartridge is correctly located such that the wick means is in direct contact with the microheater element 11 or otherwise in the correct location. Release of the vapour from the cartridge is provided through a pair of apertures located either side of the wick means in the vicinity of the microheater element 11. Alternatively, the recess may have a pair of notches or indentations, one on each side, that interference fit with a corresponding pair of projections or lugs, one on each side, on the cartridge.

Thus in use the cartridge is inserted by the user through the opening 4 to the recess via the guide means 12, which may incorporate the aforementioned lugs or notches (not shown), or the device may already be fitted with such a cartridge having a refill pack housing the chemical formulation. The user then turns the device on through the switch means 6 and may initiate a burst mode to provide additional bursts of the vapour through the vents 3. The use may be for instant personal use or be left by the user in a room, such as a bedroom, switched on in situations where insecticide is to be dispersed at regular intervals into the room over a set period of hours. The device may have a clip to be attached to a user's clothing or belt or the like. Once an indication is provided to the user through the indicator 5 that the battery is low or the microheater element is faulty then the user simply replaces the batteries or the heater element. A user may regularly check that enough chemical formulation is stored in the reservoir and if it is low or there is no formulation left then it is simply a matter of removing the cartridge from the device and replacing it with a refill cartridge, or alternatively, refilling the present cartridge reservoir.

1-22. (canceled)

23. A portable device for enabling vapourization of a chemical formulation into an atmosphere comprising:
receiving means for receiving a cartridge, the receiving means having an opening and a recess into which the cartridge is inserted;
the cartridge storing the chemical formulation;
heater means for contacting the chemical formulation and vapourizing the chemical formulation when the cartridge is inserted into the receiving means and the heater means is energised;
aperture means to enable vapourized chemical formulation to be guided therethrough into the atmosphere;
wherein the recess of the receiving means and the cartridge have means for providing an interference fit in order to locate the cartridge in a predetermined position with respect to the heater means.

24. A device according to claim 23 wherein the recess has projection means that provide the interference fit with indentation means on the cartridge and thereby gives a tactile indication to a user that the cartridge has reached the predetermined position.

25. A device according to claim 24 wherein the indentation means is one or more notches.

26. A device according to claim 24 wherein the projection means is one or more lugs.

27. A device according to claim 23 wherein the recess has indentation means that provides the interference fit with projection means on the cartridge and thereby gives a tactile indication to a user that the cartridge has reached the predetermined position.

28. A device according to claim 27 wherein the indentation means is one or more notches.

29. A device according to claim 27 wherein the projection means is one or more lugs.

30. A device according to claim 23 wherein the cartridge has a wick in contact with the chemical formulation such that when the predetermined position is reached the wick is in contact with the heater means.

31. A device according to claim 23 wherein the recess has guide means for guiding the cartridge when the cartridge is inserted into the recess.

32. A device according to claim 23 further comprising electrical circuit means in order to provide one or more pulses sequentially to the heater means so as to enable the heater means to vapourize the chemical formulation.

33. A device according to claim 32 wherein a partition divides the interior of the device into a first compartment and a second compartment.

34. A device according to claim 33 wherein the partition comprises an electrical circuit board housing an electrical circuit means.

35. A device according to claim 34 wherein the electrical circuit board is double-sided providing electrical connections to electrical components of the electrical circuit means.

36. A device according to claim 35 wherein the electrical components of the electrical circuit means are located either on one or both sides of the electrical circuit board.

37. A device according to claim 36 wherein the heater means is mounted on a side of the electrical circuit board within the first compartment, the first compartment housing the receiving means and having the aperture means so as to enable the heater means to contact the chemical formulation on the cartridge when the cartridge is inserted into the receiving means.

38. A device according to claim 32 constructed as two detachable members wherein a first member houses the receiving means and having aperture means and a second member houses a power supply.

39. A device according to claim 38 wherein the electrical circuit means is mounted on an electrical circuit board acting as a partition between the first member and the second member.

40. A device according to claim 39 wherein the electrical circuit board is double-sided.

41. A device according to claim 40 wherein the heater means is mounted on a side of the electrical circuit board in the first member to enable the heater means to contact the chemical formulation on the cartridge when the cartridge is inserted into the receiving means.

42. A device according to claim 41 further comprising switch means to activate and deactivate power supplied to the heater means.

43. A device according to claim 42 further comprising indicator means to indicate battery charge status and/or device failure.
